(19) United States
(12) Patent Application Publication
(10) Pub. No.: US 2012/0123508 A1
(43) Pub. Date: May 17, 2012

(54) Methods and apparatus for wireless control of biological tissue

(75) Inventors: Christian Wentz, Cambridge, MA (US); Edward Boyden, Chestnut Hill, MA (US)

(73) Assignee: MASSACHUSETTS INSTITUTE OF TECHNOLOGY, Cambridge, MA (US)

(21) Appl. No.: 13/295,736

(22) Filed: Nov. 14, 2011

Related U.S. Application Data
(60) Provisional application No. 61/412,954, filed on Nov. 12, 2010.

(51) Int. Cl. A61N 5/06

(52) U.S. Cl. 607/88

(57) ABSTRACT

In exemplary implementations of this present invention, a supercapacitor-based electronic device delivers high currents to an array of implantable light sources or electrodes. The device receives wireless power from an external transmit coil and receives control signals from either an onboard computer or external wireless data telemetry.

### RELATED APPLICATIONS

This application claims the benefit of U.S. Provisional Application Ser. No. 61,412,954, filed Nov. 12, 2010, the entire disclosure of which is herein incorporated by reference.

### STATEMENT REGARDING FEDERALLY SPONSORED RESEARCH OR DEVELOPMENT

This invention was made with U.S. government support under Grant Numbers NIH 1RC1 MH088182 and NIH 1R43NS070453, each awarded by the National Institute of Health. The government has certain rights in this invention.

### FIELD OF THE TECHNOLOGY

The present invention relates generally to wirelessly powered devices for control of biological tissue.

### SUMMARY

In exemplary implementations of this present invention, a supercapacitor-based electronic device delivers high currents to an array of implantable light sources or electrodes. The device receives wireless power from an external transmit coil and receives control signals from either an onboard computer or external wireless data telemetry.

This invention may be used to advantage for the optical control of neural tissue. For example, the supercapacitor-based device may deliver high currents to an array of optical fibers or an array of waveguides that is inserted into a mammalian brain, or positioned adjacent to the brain. Each fiber or waveguide in the array is coupled to a light source (LED or laser). The brain has been previously sensitized to light, using genetically encoded optical neural control reagents, which are delivered either using viruses or via transgenic means. The array is used to optically perturb the brain. For example, the neurons of the brain may be activated by one color of light, and/or silenced by another color of light.

This invention may be embodied as a wirelessly powered and wirelessly controlled headborne system capable of simultaneously driving multiple LEDs and recording neural activity in an awake behaving animal.

A prototype of this headborne system has demonstrated reliable optical stimulation in mice for greater than 3 months while weighing in total approximately 3 grams. This device negates the need for tethered stimulation systems and associated limitations on animal behavior.

In some implementations of this invention, an implanted device includes a supercapacitor for energy storage and light sources (e.g., LEDs) for optical control of biological tissue. For example, the device may be cranial implant for optogenetic control. Transcutaneous energy transfer (TET) may be employed to wirelessly deliver energy to the supercapacitor-based implanted device. The external power transmitter for the TET may be battery powered or mains powered.

Alternately, the device for optical control may be external (and located adjacent to or affixed to) the mammal. In that case, wireless TET is not employed. Instead, a wireless power transmitter (using EMF waves) delivers power to the external device. A supercapacitor is used to store energy in the device. Light sources (e.g. LEDs) are housed in this external device.

In some implementations, the device is not used for optical control of biological tissue, but is instead used for electrical stimulation or other EMF-based stimulation or perturbation of biological tissue.

In some implementations of this invention, a DC/DC converter circuit in the device is used, after rectification of wirelessly-received energy, to convert higher voltages (e.g., 10-15V open circuit) to lower supercapacitor-safe voltages (e.g., 3-6 V). This makes it easier to avoid a supercapacitor overvoltage condition.

In some implementations, the supercapacitor-based device receives wireless power from an external source continuously or intermittently (more than once every 24 hours). For example, the device may be implanted, and wireless transcutaneous energy transfer (TET) may be used to continuously power the device, without having a secondary storage device (e.g. battery). In that case, a supercapacitor housed in the implant used as a primary storage device.

In some implementations, a DC/DC converter topology before the supercapacitor is used to tune the RF power link open circuit voltage.

In some implementations, a DC/DC converter circuit before the supercapacitor steps down input voltage to a safe voltage for the capacitor (e.g., 5-15 volt input from the power receiver, stepped down to ˜5V for the supercapacitor).

In some implementations, a DC/DC converter circuit after the supercapacitor (that has either a buck/boost or charge pump topology) delivers fixed voltage and/or fixed current from the capacitor over a range of capacitor voltages.

In some implementations of this invention, light output is adaptively controlled by a processor, based at least in part on an algorithm that models heat transfer in tissue (as opposed to merely sensed temperature in tissue).

In some implementations, the processor generates control signals to shutdown light delivery if an increase in tissue temperature (as sensed or modeled) exceeds a specified threshold. This threshold may be programmable, rather than fixed (e.g., rather than a fixed threshold of 1 degree Centigrade). Also, the processor can manage (signal back externally) the state of the capacitor over wireless telemetry. This can be used either onboard or externally for algorithmically optimizing the amount of power to be wirelessly delivered to the device based on the light pulse profile (e.g., transiently increase TET power when higher powers are used by the light sources). In addition, the processor can provide remote updates of state variables (temperature, biosensing, pulse profile, supercapacitor voltage, DC/DC converter input voltage) to the system.

In some implementations, the device is employed for integrated recording of biopotentials using EEG, ECoG, or extracellular electrodes.

The power receiver antenna may be multi-axis. For example, a 3-axis receiver may be used, which allows for power reception regardless of the orientation of the receiver relative to the transmitter.

In some implementations, a multiple unit system is deployed. For example: (a) multiple animals may each have a single capacitor and optical array; (b) a single capacitor may power multiple optical arrays for a single animal, or (c) several units, each with their own capacitor, may run in different locations on a single patient that has multiple needs. In each case, a processor may output control signals to manage the different units that are running simultaneously

The above description of the present invention is just a summary. It is intended only to give a general introduction to some illustrative implementations of this invention. It does not describe all of the details of this invention. This invention may be implemented in many other ways.

### BRIEF DESCRIPTION OF THE DRAWINGS

FIG. 1 is a schematic side view of a 3 gram, 1.5 cm³ wirelessly powered and controlled headborne electronics architecture with implanted light sources.

FIG. 2 is a schematic diagram of a headborne/implant device that is powered by an external power transmitter and that employs a data telemetry link.

FIG. 3A is a block diagram of a headborne/implant system.

FIG. 3B is a perspective view of the underside of an optics module.

The above Figures illustrate some illustrative implementations of this invention, or provide information that relates to those implementations. However, this invention may be implemented in many other ways. The above Figures do not show all of the details of this invention.

### DETAILED DESCRIPTION

The following is a description of an illustrative prototype of this invention.

In this prototype, under continuous optical stimulation, an implant can deliver >2 W of power, sufficient to drive two 700 um×700 um LEDs at 100% duty cycle at programmable frequency indefinitely (e.g., ˜500 mW/mm̂2 to cortical targets), while recording from as many as 4 electrodes simultaneously. This is a very high level of power; for surface LEDs atop cortex, dozens can be run indefinitely. Intermittent LED input power can be increased to 5 W for one second, enough to drive 5 LEDs at 100% duty cycle or 10 LEDs at 50% duty cycle. Intermittent high power delivery above the wirelessly supplied 2 W is achieved using an onboard supercapacitor energy storage cell with >4 Joules of reserve energy, thus a scaling law for burst mode power of 2 W+4 W*seconds/duty ratio. This prototype supports a maximum of 2 driven LEDs at any time, though additional driver channels are easily added with a nominal increase in weight.

In this prototype, wireless power is delivered at low magnetic field (400 A/m) and low frequency (120 kHz) to reduce the possible side effects of magnetic field exposure. Power transfer is maintained at high efficiency (10%) by utilizing a synchronized bridge driving circuit and precision frequency tuning. The receiver is also optimized for power efficiency over a wide range of the animal's body orientation by utilizing an axial resonant LC receiver coil with high permeability core and under-cage transmit coils. The power receiver circuit is optimized to produce a maximum of 2 W of received power, achieved at a height of approximately 2 cm above the cage floor.

In this prototype, the modular design of the implant includes four distinct, removable subsystems: (i) a skull-fixed LED array with thermal sink, optional fiber light guides and optional 4-channel electrode pre-amplifier with 16-pin universal connector, (ii) an implant motherboard with LED drivers, power management stages and ultra-low power 16 MHz integrated microprocessor (Texas Instruments® MSP430), (iii) 2.4 GHz data telemetry link (TI®/Chipcon® CC2400), and (iv) wireless power rectifier. When not in use, components (ii-iv) can be removed from the head and subsystems can be upgraded, repaired or interchanged with other implanted LED arrays without significant disturbance to the animal.

The implant motherboard comprises an array of bare LED dies embedded in a thermally dissipative ˜1 gram copper block, which is affixed to the skull over a cranial window using 3 standard skulls crews and anchoring dental cement. A miniature printed circuit board (PCB) features wire-bonded traces from the LED control terminals to a 10-16 pin connector accepting the implant motherboard stack.

Signal conditioning electronics are embedded in the implant PCB, composed of four 10× gain common-referenced differential preamplifiers, band-pass filter and 10-100× gain (hardware adjustable) second stage amplifier. Thus, the motherboard and telemetry stack is capable of simultaneously record from 4 dc-coupled electrode channels at up to 25 k samples per second (ksps) and 10 bit resolution, low-pass filtered at 10 kHz.

Advantageously, as many as 83 separate implant-to-computer links can be simultaneously opened, by taking advantage of a Gaussian frequency-shift keying (GFSK) encoding scheme in the onboard radio and automatic frequency hopping algorithms in the ISM (Industrial, Scientific, Medical) 2.4-2.4835 GHz band. A flexible software layer on the radio allows for addressing of 2¹⁶ distinct implants, making this paradigm suitable for institution-scale high-throughput testing. Wireless communication with an implanted device can be added to any PC using a small USB dongle, each dongle supporting one full-speed implant link. Rapid modification to stimulation, recording and communication protocols is possible due to a flexible and open source C/C++ based software architecture running on the implant and PC-side systems. Real-time remote triggering of stimulation and recording systems is supported, allowing for complex closed-loop behavioral tasks and high-throughput screening of large animal cohorts.

### Power Systems

In this prototype, power delivery to the headborne device is achieved via precisely tuned resonant inductive coupling between matched LC networks. On the receiver side, AC signal is rectified using a full-wave passive rectifier and supercapacitor energy storage element, serving to both filter AC ripple and provide reserve energy storage. This supercapacitor element improves device reliability under varying coil-to-coil mismatch angle as the animal moves around the cage, and also allows for reserve energy to be used in high-powered short duration pulsing of the implanted LEDs. Passive Zener diode shunt and active pulse width modulated (PWM) rectifier load regulation are used to maintain safe voltage range on the supercapacitor.

Following this first stage of AC/DC conversion, a switched mode buck/boost converter (Texas Instruments® TPS61202) provides low ripple conversion from the 5.5V nominal supercapacitor voltage to a regulated 3.3V digital supply (hardware adjustable, 1.8-3.6V), serving the onboard microcontroller (MCU) and both analog and digital supplies of the radio telemetry chipsets (CC2400/2500). An additional low-dropout 1.8V linear regulator, running off the 3.3V supply, provides analog power to the radio using a chip-scale packaged device (Analog Devices® ADP121). This secondary voltage supply provides extreme flexibility in future radio chipset modifications or additional peripheral circuitry.

A second buck/boost converter is utilized as a parallel LED driver circuit to power the implanted LEDs. This circuit runs directly off of the supercapacitor, with capability to operate at voltages as low as 0.3V, making it ideal for potentially variable input powers and output loading.

### Resonant Power Coupling and Supercapacitor Energy Storage

In this prototype, a 6″ diameter under-cage transmit coil with series capacitive elements and 3 mm diameter, 15 mm long axially wound coil with ferrite core and parallel capacitive element on the head of the animal delivers 2 W of output power to the headborne device. These power levels are achieved in a low strength magnetic field of 400 A/m oscillating at 120 kHz.

Rectification of the coupled AC signal on the headborne device is performed with a full-wave passive rectifier using low forward voltage Schottky diodes and a supercapacitor filtering element of 22-150 mF.

The choice of energy storage method here is dominated by the need for rapid charging and discharging capability. Instantaneous LED currents in this prototype are on the order of 400 mA or more. A typical stimulation waveform for treatment of a Parkinson's Disease model may require 5 ms pulse width, 130 Hz stimulation, equating to a duty cycle of approximately 70%. Ultimately, this requirement suggests a preference for high power density devices rather than a strictly high energy density solution, mitigating temporary bursts of stimulation over the averaged power capability of the system, or in the case of intermittently poor power coupling.

In this prototype, a supercapacitor—rather than lithium ion rechargeable cells—is used, for the following reasons: Lithium Ion rechargeable cells are an attractive option at certain scales given their substantial energy density of ˜620 J/gram, typical peak discharge rates of approximately 5 C and pulsed rates as high as 25 C. However, several limitations to Li-Ion cells can make them disadvantageous in this design. Generally, Li-Ion batteries must be carefully regulated to prevent deep discharge below 2.0V or overcharge above 4.2V. Moreover, higher power density batteries and smaller batteries in general have a higher volume fraction dedicated to non-storage contributing elements like current substrates, thus limiting their practical energy storage. 400 mA discharge currents required by this design imply a 80 mA-hour cell to maintain safe discharge rates of 5 C, while only a few Joules of energy storage is required to sustain surge currents experienced in photostimulation of neural networks in vivo. A one second pulsed sequence on an array of 5 LEDs operating at nominal 400 mA, 3.6V load requires 7.2 J of energy, less than 5% of the energy supplied by an 80 mA-hour batter maintained between 4.2 and 2.0V. Thus, a Li-Ion cell is highly stressed in power density yet underutilized in energy density.

By contrast, supercapacitors can achieve on the order of 10 J/gram energy density, but can sustain discharge currents of 1000 C, implying that a one gram ideal supercapacitor satisfies the demands of this system, while serving the additional purpose of filtering rectifier output. In practice, packaging dominates the weight and volume of these small supercapacitors.

In this prototype, a supercapacitor is used for energy storage. A prototype thin-film solution was chosen due to its low ESR (equivalent series resistance) and 5.5V limit (CAP-XX®, 300 mF, 5.5V, ESR 70 mOhm).

The ESR limit tends to be the limiting factor in terms of the maximum usable output current of most supercapacitors at the small scale (a few grams), particularly at the 120 kHz frequency operation of this prototype.

### Power Converters and LED Driver

In this prototype, two DC/DC converter subsystems are present: one system (Texas Instruments® TPS61202) is dedicated to providing efficiently regulated 1.8-3.6V digital supply for the onboard MCU and sensitive RF chipset, and another (either the Texas Instruments® TPS61150 or another TPS61202) for high power up/down-conversion to drive the implanted LED array.

Both of these systems utilize off-chip inductors of 2-10 uH and input/output filtering capacitors of approximately 10 uF. Shielded inductors were chosen in both instances to minimize EMI to the RF and recording systems.

A parallel, high-side switched multiplexing topology is employed, using MCU-addressed BJTs to select which LEDs were to be driven at any time.

Advantageously, a voltage-controlled LED driver allows for substantially great output currents to be achieved. For example, with a 5.0V nominal supercapacitor supply, efficiencies of 90% and maximal output currents of 1.8 A are achievable at a fixed 3.5-3.9V LED forward voltage. This rating equates to 4.5 LEDs driven at 400 mA, or 7 W of output power.

Voltage-mode control does, however, result in exponential output power variation with LED forward voltage. Moreover, LED output power varies significantly with junction temperature, thus feedback is required if one desires to tightly regulate the output power of the implanted array; support for temperature sensing has therefore been built into the device. An LED under continuous operation may shift its forward voltage down by as much as 400 mV from ambient operating temperature. This 400 mV shift correlates to a change of perhaps 200 mA or >70% reduction in forward current. Thus, it is desirable to consider a nominal operating temperature when designing the LED driver circuit—in this prototype, a nominal 4V is chosen for drive voltage.

For the overall power management system of this prototype, an optimal value for energy storage exists such that the device is capable of performing a cold start—that is, the device may be placed in the presence of a 400 A/m drive field with all converter stages powered on, and sufficient storage capacitance exists to supply the initial startup energy for these buck/boost converters—yet does not have unnecessary amounts of reserve power (recall, the design specification was intended to provide 2 J of bursting energy). An analytical solution based on the load currents of a typical LED, inductor and input and output capacitor sizing would suggest a nominal supercapacitor of just a few tens of millifarads in addition to the amount of reserve power required (in the 2 J case, roughly 66 mF), but bench testing suggests 2-3× this amount of energy is necessary, largely due to the buck converter's input surge current. Thus, a 300 mF supercapacitor was used for this prototype. If a smaller capacitor is necessary, the system may be powered initially off of the 1.8V linear regulator. Once supercapacitor voltage reaches full charge, the buck/boost stages can then be enabled without substantial capacitor voltage droop.

### Far-Field Wireless Link

In this prototype, a headborne device features a 1 Mbps 2.4-2.4835 GHz ISM band wireless transceiver based upon the TI®/Chipcon® CC2400 integrated chipset with off-chip oscillator and differential to single-ended balun with chip antenna. An 8-pin header with 3-wire SPI interface to the MCU, 2 auxiliary I/O pins, 1.8, 3.3V and ground terminals allows for independent re-design of the wireless transceiver.

The MSP430F2132 MCU runs a simple three state interrupt-based Finite-State Machine to minimize idle transceiver on-time. Transition delay between the three states, Transmit, Receive and Idle is adjustable in software in this implementation. Alternately, a channel-adaptive delay may be programmed onto the MCU to further improve performance.

### System Control Architecture and Onboard Processor

In this prototype, an onboard integrated 16-bit, 16 MHz microcontroller (TI MSP430F2132) with 8-channel, 200 ksps 10-bit analog-to-digital converter (ADC), 512 byte RAM and 264 kB flash memory supervises on-the-head computation. This chipset is used to sample analog signal from the 4-channel neural recording amplifiers and optional temperature and supercapacitor voltage sensors, enable data transmission to and from the wireless telemetry, and handle pulse width modulation (PWM) of the LED drivers to in turn modulate neural network activity in vivo.

In this prototype, the overall control architecture is as follows:

Initialization: Upon startup, the MCU transmits 44 data frames of 24 bits each to initialize the radio chipset. By default this initialization is at 2.4000 GHz transmit frequency, Gaussian Pulse Shift Key (GPSK) modulation, 1 MHz channel width, and 1 Mbps data rate via unbuffered transmit mode. A CRC is, however, calculated.

Channel ID: The remote device transmits its unique channel ID periodically at default frequency of 2.4000 GHz, awaiting instruction from the PC-side controller, implemented as a simple Hyperterminal interface to a USB-connected 2.4 GHz transceiver.

Control Signals: Control signals from the PC-side controller trigger state change in the remote device—4 Neuromodulation parameters, namely LED address, pulse frequency, pulse width and on/off toggle—and 3 Recording parameters, namely electrode channel select, sample rate and on/off toggle are all transmitted to the remote device with unique device ID. In addition, optional LED temperature and supercapacitor voltage monitoring can be toggled, to have the remote device transmit real-time state variables to the PC. These 9 control variables may be updated at any time by text entry into the Hyperterminal window. Finally, a frequency hopping routine can be initiated to find the nearest clear channel in the ISM band.

Neuromodulation Programming: On the remote device, these pulse frequency and pulse width parameters are stored as 16-bit comparator trigger values. An up/down waveform generator operating at 1.2 MHz is toggled on and off by the on/off toggle control signal, such that the comparator output triggers the particularly addressed LED on/off with a square wave. This allows for independent control of two LED waveforms simultaneously, and 8-LED addressing using a direct port-to-port mapping of pins. Alternately, an addressing scheme may be implemented using a binary addressing scheme.

Pulse Frequency Programming: Frequency is transmitted to the device as a decimal number (e.g. ‘FR=130’=130 Hz). On the device, decimal to hex conversion is performed, and the upper trigger register (TACCR0) is set as TACCR0=Clock frequency/2*Pulse frequency.

Pulse Width Programming: Pulse width parameter is transmitted as a decimal number in milliseconds (e.g. ‘PW=5’=5 ms). On device this is converted to hex, and stored in the Toggle/Set register (TACCR1 for LED channel 1, TACCR2 for LED channel 2), as TACCR1/2=PW*Clock frequency*500.

Recording Programming:—On the remote device, the electrode channel select and sample rate are programmed into a direct memory access (DMA) controller which automates the sampling of data from the 10-bit ADC to memory or flash. By default, data is written temporarily to memory and then output unbuffered to the radio for streaming recording.

Supercapacitor Overvoltage Control:—Active PWM control of supercapacitor voltage is employed. The MCU's comparator's interrupt flag is enabled to alert the system in the event that voltage on the supercapacitor exceeds safe 5.5V threshold. In this instance, the MCU open circuits the rectified in a back-off-and-wait manner for a programmed period of time.

Channel Frequency Hopping:—In modes of operation where either multiple animals are under simultaneous control, an optional channel hopping protocol can be initiated between PC-side and remote device to find the nearest clear channel.

### Optics

In this prototype, the headborne device can be easily disconnected from an implanted optics array. This array comprises a set of bare die LEDs affixed to a small copper block, which serves as thermal sink to draw lost energy away from the brain. LEDs are wire bonded to a small PCB also connected to the copper block, which serves as an anchor point for a 10-16 pin Samtec® connector allowing easy disconnect of the headborne electronics for repair or replacement, to ease animal housing needs, etc.

In the event that deep brain structures are targeted for optogenetic control, small fiber waveguides are affixed directly to the surface of the LED die using optics glue, and the remaining die surface coated with reflective epoxy. An additional reflector is placed behind the die for secondary redirection. Significant loss of energy results from this arrangement due to the uncollimated nature of LED light. Alternately, lasers may be used as light sources to reduce these energy losses. Still, with the LEDs in this prototype, output power at the tip of the fiber is approximately 5-10 mW/mm̂2, sufficient for local activation of ChR2 targeted neurons.

### Sensing

In this prototype, an integrated 8-channel 10-bit ADC affords the ability to monitor critical device parameters remotely. This feature is useful for diagnostic purposes in the event of aberrant behavior on the part of the animal, and also adds an element of safety to the device by allowing remote shutdown in the event of supercapacitor overvoltage, over-current (indicating a short circuit), or over temperature events. Many of these features are also hard-wired into the existing circuitry, e.g. over-temperature shutdown in the TPS61202 and over-current limiters. Specific designs are listed below.

### Rectifier Current Monitor

In this prototype, rectifier current is monitored. This monitoring is useful not only for diagnostic purposes, but also in the event that alternative power transfer techniques are employed. Magnetostrictive/Electroactive (ME) sandwich materials for power conversion can be employed. In such designs, resonant frequency is tightly coupled to the temperature of the core material. As such, a constant monitor of rectifier current can be used to optimize drive coil tuning frequency over a wide range of implant temperatures.

In this prototype, a high-side current shunt monitor is used to measure small voltage drops across a precision sense resistor placed in series with the supercapacitor load. The shunt monitor (Texas Instruments® INA193) amplifies the voltage drop across a 0.3 Ohm sense resistor by a factor of 20-100 V/V and outputs this voltage to one of the eight ADC channels with operating range of Vdd. This implies a maximal sensed current of 500 mA for 3.3V supply, sufficient for a 2.5 W rectifier with 5.0V nominal capacitor voltage. Power loss due to the sense resistor is Î2*Rsense=75 mW. Reduction of sense resistor below 0.3 Ohms linearly reduces power loss, though values much below this require careful layout to achieve accurate readings. Alternately, a current sense FET may be used (which may be advantageous in higher power settings or where dissipation is critical).

### Supercapacitor Voltage Monitor

As already described, supercapacitor peak voltages are generally limited to only a few volts before breakdown occurs. Catastrophic breakdown is very easily achievable with the power figures developed in this system.

Thus, in this prototype, a first order protection circuit implements a current shunt. The 5.5V peak allowed by the supercapacitors is convenient for use of a Zener diode at 5.1 or 5.6V, though care must be taken to ensure that the diode is rated for peak power dissipation of several watts. An active monitoring loop is also implemented, in which an N-channel depletion mode transistor (FET) is placed in the conduction path from rectifier to supercapacitor. The depletion mode FET normal conducts when gate voltage is zero, allowing the supercapacitor to charge even though the digital control loop is not yet powered on. Once supercapacitor voltage is high enough for the digital supply to turn on and initialize the microcontroller, the microcontroller begins actively sampling supercapacitor voltage. If voltage sampled is above the threshold voltage (5.5V) for safe operation, a positive gate voltage is applied to turn off the control FET for a fixed duration, defined in a “back off and wait” paradigm.

### Recording

A recording amplifier with 4 input channels, one serving as common mode ground, is built into the implant headstage motherboard. The first stage amplifier is a unity to 20× gain, capacitively coupled single rail amplifier based upon the Texas Instruments® TLC2264 quad operational amplifier. All four channels including the reference channel are buffered in this manner. With the first channel serving as common ground, the outputs of the first stage are fed into a 10-1000× programmable gain instrumentation amplifier (INA333) with first order low pass filter set to approximately 10 kHz. The input impedance of the system is approximately 10¹² Ohms. Adjustment of the second stage gain is done in hardware, by replacing the gain setting resistor on each of the INA333's. Advantageously, these amplifiers are easily swappable given the modular design, such that any preamplifier producing a controllable gain output voltage can be utilized.

### Thermal Considerations

Thermal dissipation of electronic components on the head is a significant concern. In this prototype, numerous design principles have been employed to limit the amount of unwanted heat generation. Where possible, dissipative elements (sense resistors, diodes, pass transistors) have been chosen to reduce conduction loss. The microcontroller and radio elements can operate in a low power mode, with rapid startup times on the radio oscillator core and the MCU.

### PC-Based Device Interface

In this prototype, a modified USB-to-serial JTAG programmer serves as a base for a wireless base station for communication with the remote device.

The USB programmer/wireless base station has a 16-pin Samtec® header for mounting an implant motherboard and radio PCB. With a simple re-flashing of memory, the implant becomes a 1 Mbps tether. The original TI MSP430-UIF programmer (red PCB) has been modified to allow for full speed communication with the USB port, as default units are limited to 9600 baud backchannel communication.

In this prototype, all PC-side interface software is maintained in firmware in the tethered device, thus, no software other than a Windows Hyperterminal is required. The USB device reports itself as a serial interface with a dedicated COM port. The user connects the USB device, opens a new Hyperterminal session using the COM port associated with the USB port on their PC, and the tethered device automatically initializes with a splash screen listing the command-line options for communication with tethered devices. All keystrokes are parroted back to the user, such that the link appears to be native software.

In this prototype, a USB-connected wireless tether unit is employed. A modified JTAG interface microcontroller programmer is used to open a serial COM port between a docked electronics stack and a host PC. The microcontroller handles all network protocols to communicate with up to 83 devices simultaneously on ISM 2.4 GHz band in 1 MHz channel spacing, and communicates with the PC using standard UART protocol. A 16-bit identifier system allows up to 2̂16 different devices to be controlled using intermittent polling.

### Behavioral Demonstration

To demonstrate the high power capacity of the headborne wireless optical stimulator, a Parkinson's Disease (PD) mouse model was wirelessly treated using a simple, autonomous version of this invention. A headborne system was pre-programmed to generate a 130 Hz, 5 ms pulse width waveform previously reported to successfully halt Parkinson's like behavior in the PD mouse model. The device was targeted toward right hemisphere M2 motor cortex, some of whose afferent axons projection to the subthalamic nucleus, a target known to be effective in deconstruction of Parkinsonian essential tremor. The PD behavioral phenotype is modeled as a rightward tendency to rotate on the part of the animal. When stimulated with the wireless system, the animal shows a halting of rotational behavior. This behavior has recently been demonstrated in fiberoptically tethered animals, however, the power requirements of stimulation, roughly 1 W input power to the LED, previously made wireless operation inaccessible.

### More Details

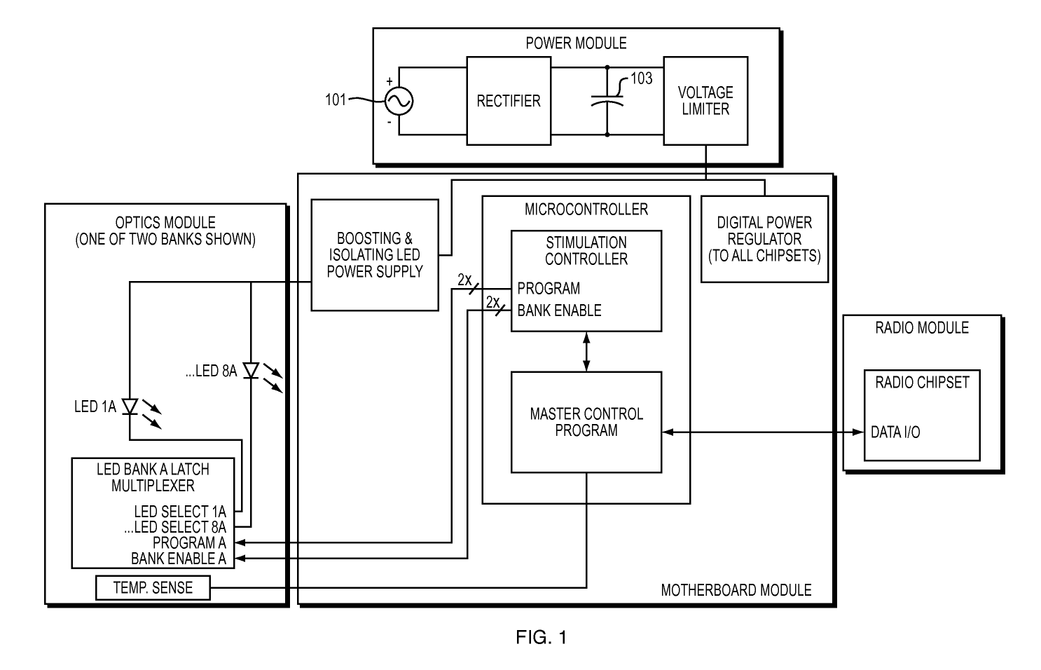

FIG. 1 is a schematic side view of a 3 gram, 1.5 cm³ wirelessly powered and controlled headborne electronics architecture with implanted light sources, in a prototype of this invention. The optics module, housing light sources (LEDs), temperature sensing and LED multiplexers are affixed to the skull, while other modules are detachable. A motherboard module, with embedded microcontroller, LED driver and power management circuitry, delivers pulse waveforms to implanted light sources via 6-pin connector. Additional pins allow for in-the-field reprogramming of electronics stack with USB computer adapter. A power module includes a power antenna 101 and a supercapacitor 103. The power module connects to motherboard using 3-pin connector, and contains wirelessly coupled power from under-the-cage transmitter. Passive and active voltage limiting circuitry protects from potentially damaging over-voltage events. A minimal configuration without radio module allows for chronic delivery of pre-programmed waveforms for open-loop neuromodulation experiments. Finally, real-time continuous monitoring and control of up to 83 headborne devices in a given shared radio space is enabled with 1 Mbps radio module using a standard PC with USB-connected wireless dongle. Thousands of devices may be intermittently addressed using polling schemes, allowing for high throughput behavioral research.

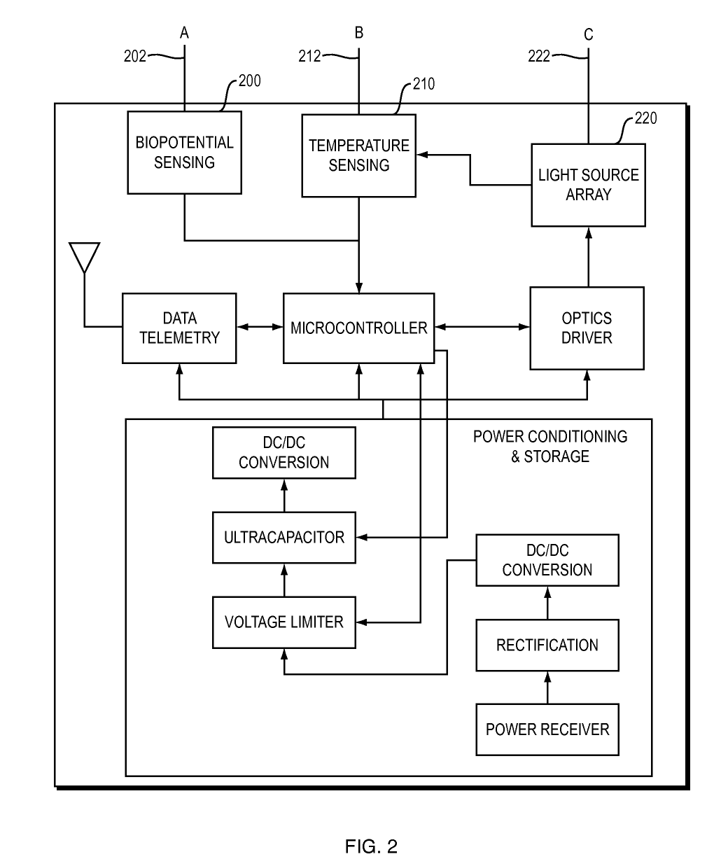

FIG. 2 is a schematic diagram of a headborne/implant device that is powered by an external power transmitter and that employs a data telemetry link. The device comprises the items within the outermost box in FIG. 2. Biopotential sensing amplifiers 200 are linked by a wired or wireless connection 202 to electrodes (not shown) near the headborne or implantable device for closed-loop operation of the light source array. Internal temperature sensor 210 is linked by a wired or wireless connection 212 to nearby tissue (not shown) for closed loop operation of a light source array safety cutoff. Light from a light source array 220 is transmitted by a transmission means (e.g., fiber optic or lens) 222 to nearby tissue (not shown).

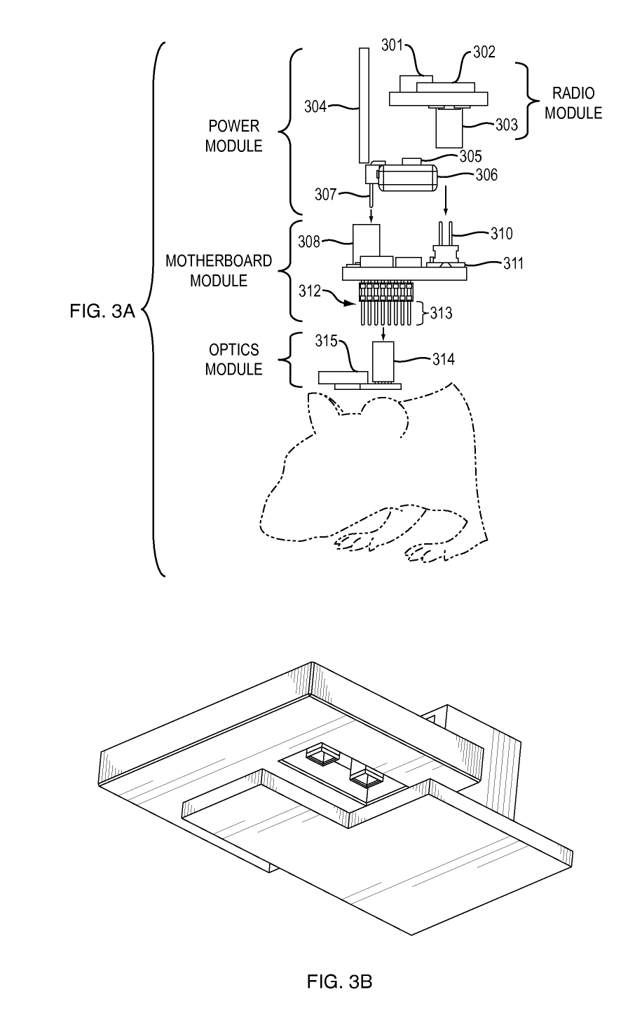

FIG. 3A is a block diagram of a headborne/implant system, in an illustrative implementation of this invention. Four total modules are represented: Power management, Optics, Motherboard and Radio modules. Wireless power is rectified and stored using onboard supercapacitor, enabling intermittent bursts of 5 W of power to optics module. Up to 16 LEDs, grouped into two 8-LED banks are independently addressable using latch circuitry. Banks may be controlled separately by two different control waveforms. Radio features high-speed 1 Mbps wireless data link for realtime remote control of hundreds of headborne units. As shown in FIG. 3A, the headborne system includes a radio antenna 301, radio transceiver chipset 302, female radio receptacle 303, power receiver antenna 304, power rectifier circuitry 305, supercapacitor 306, male power connector 307, motherboard power receptacle 308, microcontroller 309, male radio connector 310, analog power buck/boost converter 311, male interface connector 312, male programming interface pins 313, female optics interface receptacle 314, and LED thermal sink 315.

FIG. 3B is a perspective view of the underside of an optics module, in an illustrative implementation of this invention.

### Some Applications

In illustrative implementations, this invention has many practical applications: Among other things, the wireless optical neural control system may be used for in vivo neuroscience research. The freedom to explore complex environments while maintaining recording and optical neuromodulation capability (which the present invention allows) is highly desirable. The ability to remotely address dozens of device simultaneously presents an opportunity to perform high-throughput screening of complex behaviors. Whereas previously researchers were required to select the animal, tether the animal singly, perform the experiment and begin anew, such research can become at least semi-automated. Additionally, closed-loop paradigms in complex environments, in which an optogenetic control event is triggered based upon either neural network activity or behavioral event, can be easily implemented without modifying the hardware or software interface already developed.

### Variants

This invention may be implemented in many different ways, and is not limited to the prototype implementation described above. Here are some non-limiting examples:

An alternative embodiment of this invention includes a highly miniaturized application-specific integrated circuit (ASIC) with RF telemetry system, microcontroller, low channel count neural amplifier and light source controller on a single piece of silicon, with thin-film supercapacitor, solid state laser element and power and data antennas all packaged in a sub-millimeter scale device. Such an assembly is similar in function and system architecture, but through miniaturization using standard VLSI and thin film processing techniques art, the miniaturized device may be injected into target brain regions in the human. Independent devices can be programmed with different stimulation waveforms stored in microcontroller memory to modulate differing brain regions with spatiotemporal specificity. Closed-loop algorithms may be implemented by sensing on the integrated neural amplifier and delivering the appropriate optical modulation signal algorithmically. A single external power transmitter can safely power all devices using frequencies in the sub-MHz band. Using such a system, whole-brain modulation can be employed without the need for a bulky implant and large numbers of long-distance optical fibers.

Different types of power sources may be used in this invention. For example, the supercapacitor may have a thin-film, tantalum, or electrolytic wet cell design. The supercapacitor design may employ series stacking of matched elements. The number of supercapacitors may vary. One or more supercapacitors may be employed. Alternately, batteries may be used, although these may have disadvantages in many applications, as discussed above. Alternately, a wired power source may be employed, although this may require a tethered or immobile subject.

Light sources other than LEDs may be employed. For example, lasers may be used.

In some implementations of this invention, thermistor-based temperature monitoring circuits are employed. For example, a simple resistor divider circuit using a remotely located thermistor epoxied to the LED heat sink, in series with a bias resistor, can be placed across the input to one of the ADC channels. Capacitive filtering can be added, using the input resistance of the ADC to create a high-pass filter pole. (Such capacitive filtering can be employed for all voltage-based sensing used in the system). Also, for example, the LED drive systems can incorporate junction temperature compensation.

This invention may be implemented as apparatus for optical control of tissue of a living organism, which apparatus comprises: (a) at least one supercapacitor for energy storage; (b) one or more light sources, (c) an array of optical fibers or light guides for delivering light from the one or more light sources to the tissue; and (d) an antenna and circuitry for receiving power by wireless transmission from an external transmit coil. Furthermore: (1) the apparatus may be adapted for implant in the living organism; (2) the wireless transmission may comprise transcutaneous energy transfer; (3) the apparatus may be adapted for cranial implant and the living organism may be a mammal; (4) the apparatus may be adapted to be positioned adjacent to an exterior surface of the living organism, in a position such that the one or more light sources are partially or wholly located externally to the living organism and the array is at least partially inserted into the living organism; (5) at least some of the one or more light sources may comprise light emitting diodes; (6) the tissue may be neural tissue; (7) the apparatus may further comprise sensors for recording neural activity in the organism; (8) the apparatus may further comprise a 3-axis power receiver antenna, (9) the apparatus may further comprise a DC/DC converter for reducing voltage of wirelessly-received energy, after rectification and before delivery to the supercapacitor; (10) the supercapacitor may store energy, which energy is received wirelessly from an external source continuously or more than once every 24 hours; (11) the apparatus may further comprise a DC/DC converter for tuning RF power link open circuit voltage; (12) the apparatus may also comprise a DC/DC converter circuit for delivering an output voltage over a range of capacitor voltages, which output voltage does not vary more than 15%, or for delivering an output current over a range of capacitor voltages, which output current does not vary more than 15%; (13) at least part of the converter circuit may have either a buck/boost or charge pump topology; (14) the apparatus may further comprise a processor for adaptively controlling light output from the one or light sources, based at least in part on an algorithm that models heat transfer in the tissue; (15) the apparatus may further comprise a processor for generating control signals to shutdown light delivery if an increase in tissue temperature exceeds a specified threshold; (16) the processor may accept user input to change the specified threshold; and (17) the apparatus may further comprise one or more sensors for measuring biopotentials.

This invention may be implemented as an implant device for implantation into a living organism, which implant device comprises at least one supercapacitor for energy storage, one or more electrodes for electrical stimulation of tissue of the living organism, and an antenna and circuitry for receiving power by wireless transmission from an external transmit coil by transcutaneous energy transfer. The implant device may further comprise a processor for generating control signals to reduce power dissipation if an increase in tissue temperature exceeds a specified threshold.

### SOME DEFINITIONS AND CLARIFICATIONS

Here are some definitions and clarifications of some terms. As used herein:

A “supercapacitor” (or an “ultracapacitor”) refers to a capacitive energy storage device of at least 100 millifarads. For example, an electrolytic double-layer capacitor of at least 100 millifarads is a “supercapacitor”, as that term is used herein.

The term “light guide” includes a microfabricated wave guide for delivering light

The term “or” is an inclusive disjunctive. For example “A or B” is true if A is true, or B is true, or both A or B are true.

The term “include” shall be construed broadly, as if followed by “without limitation”.

### CONCLUSION

It is to be understood that the methods and apparatus which have been described above are merely illustrative applications of the principles of the invention. Numerous modifications may be made by those skilled in the art without departing from the scope of the invention. The scope of the invention is not to be limited except by the claims that follow.

What is claimed is:

1. Apparatus for optical control of tissue of a living organism, which apparatus comprises:
at least one supercapacitor for energy storage,
one or more light sources,
an array of optical fibers or light guides for delivering light from the one or more light sources to the tissue, and
an antenna and circuitry for receiving power by wireless transmission from an external transmit coil.

2. The apparatus of claim 1, wherein the apparatus is adapted for implant in the living organism.

3. The apparatus of claim 2, wherein the wireless transmission comprises transcutaneous energy transfer.

4. The apparatus of claim 1, wherein the apparatus is adapted for cranial implant and the living organism is a mammal.

5. The apparatus of claim 1, wherein the apparatus is adapted to be positioned adjacent to an exterior surface of the living organism, in a position such that the one or more light sources are partially or wholly located externally to the living organism and the array is at least partially inserted into the living organism.

6. The apparatus of claim 1, wherein at least some of the one or more light sources comprise light emitting diodes.

7. The apparatus of claim 1, wherein the tissue is neural tissue.

8. The apparatus of claim 7, wherein the apparatus further comprises sensors for recording neural activity in the organism.

9. The apparatus of claim 1, further comprising a 3-axis power receiver antenna.

10. The apparatus of claim 1, further comprising a DC/DC converter for reducing voltage of wirelessly-received energy, after rectification and before delivery to the supercapacitor.

11. The apparatus of claim 1, wherein supercapacitor can store energy, which energy is received wirelessly from an external source continuously or more than once every 24 hours.

12. The apparatus of claim 1, further comprising a DC/DC converter for tuning RF power link open circuit voltage.

13. The apparatus of claim 1, further comprising a DC/DC converter circuit for delivering an output voltage over a range of capacitor voltages, which output voltage does not vary more than 15%, or for delivering an output current over a range of capacitor voltages, which output current does not vary more than 15%.

14. The apparatus of claim 14, wherein at least part of the converter circuit has either a buck/boost or charge pump topology.

15. The apparatus of claim 1, wherein the apparatus further comprises a processor for adaptively controlling light output from the one or light sources, based at least in part on an algorithm that models heat transfer in the tissue.

16. The apparatus of claim 1, wherein the apparatus further comprises a processor for generating control signals to shutdown light delivery if an increase in tissue temperature exceeds a specified threshold.

17. The apparatus of claim 16, wherein the processor can accept user input to change the specified threshold.

18. The apparatus of claim 1, wherein the apparatus further comprises one or more sensors for measuring biopotentials.

19. An implant device for implantation into a living organism, which implant device comprises at least one supercapacitor for energy storage, one or more electrodes for electrical stimulation of tissue of the living organism, and an antenna and circuitry for receiving power by wireless transmission from an external transmit coil by transcutaneous energy transfer.

20. The implant device of claim 19, wherein the implant device further comprises a processor for generating control signals to reduce power dissipation if an increase in tissue temperature exceeds a specified threshold.
